(19) United States
(12) Patent Application Publication
(10) Pub. No.: US 2021/0177735 A1
(43) Pub. Date: Jun. 17, 2021

(54) COSMETIC COMPOSITION INCLUDING CURCUMA AROMATICA SALISB EXTRACT AND ELAEOCARPUS SYLVESTRIS VAR. ELLIPTICUS EXTRACT FOR BLOCKING BLUE LIGHT

(71) Applicant: FARMSKIN INC., Cheongju-si (KR)

(72) Inventors: Taeil KWAK, Cheongju-si (KR); Kwang II KIM, Seoul (KR); Junhyuk KIM, Seoul (KR); Hyun-Chul PARK, Cheongju-si (KR); Jaehyeok CHOI, Cheongju-si (KR)

(21) Appl. No.: 17/085,315

(22) Filed: Oct. 30, 2020

(30) Foreign Application Priority Data
Dec. 12, 2019 (KR) 10-2019-0166094

(51) Int. Cl. A61K 8/9794; A61K 8/9789; A61Q 17/04

(52) U.S. Cl. CPC A61K 8/9794; A61Q 17/04; A61K 8/9789

(57) ABSTRACT

The present disclosure relates to a pharmaceutical composition for prevention, improvement or treatment of light-derived skin cell damage, a health supplement food composition for prevention or improvement of light-derived skin cell damage and a blue light blocking cosmetic composition, each of which includes Curcuma aromatica Salisb extract and/or Elaeocarpus sylvestris var. Ellipticus extract as effective ingredients, as well as a method for preparing the Curcuma aromatica Salisb extract and the Elaeocarpus sylvestris var. Ellipticus extract. Such compositions may absorb blue light having a wavelength of 380 to 500 nm, so as to protect skin cells from being damaged by blue light.

### RELATED APPLICATIONS

This application claims priority to Korean Patent Application No. 10-2019-0166094, filed on Dec. 12, 2019 in the Korean Intellectual Property Office, the entire disclosure of which is incorporated herein by reference.

### BACKGROUND

### 1. Field

The present disclosure relates to a blue light blocking composition and a pharmaceutical composition for prevention, improvement or treatment of light-derived skin cell damage due to blue light.

### 2. Description of the Related Technology

Skin is the biggest organ in a human body, which is exposed to an outside and serves to protect the body from external stimulations.

Among various external stimulations, light-derived damage due to sunlight is mostly well known. However, in recent years, there are diverse studies on harmfulness of blue light caused by displays of electronic devices such as TVs, computers, smartphones, and the like, as well as LED lighting devices (Liebmann J., et al., J. Invest. Dermatol. 130(1), 259-269 (2010); Nakashima Y., et al., Free Radic. Bio. Med. 108, 300-310(2017)). Blue light is a light of blue color in the visible light region, specifically, a light having a wavelength of 380 to 500 nm with a high energy. Since the blue light penetrates into a skin layer more deeply than UV of the sunlight, reactive oxygen species (ROS) are generated. Further, it has been reported that damage to mitochondria or DNA of cells, dysfunction of cells, cell aging, or the like are caused by the blue light (Godley B F et al., J. Biol. Chem. 280 (22), 21061-21066 (2005)).

Free radicals directly or indirectly generated from cells can oxidize protein, lipid, etc. of skin cells or destruct DNA, which in turn may cause cell apoptosis or skin cancer (Kang, S., et al., Am. J. Pathol., 166, 1691-1699(2005); Matsumura, Y., at al., Toxicol. Appl. Pharmacol., 195(3), 298-308 (2004)).

In general, intracellular antioxidant enzymes generate mutual interaction to protect cells from oxidative damage. Oxidative stress occurs when free radicals exceed interaction abilities between the intercellular antioxidant enzymes. It was reported that such oxidative stress play an important role in pathogenic process of inflammation and light-derived damage (Simon R. et al., 49, 1603-1616 (2010); Afaq F., et al., Experimental Dermatology, 15, 678-684 (2006)). Accordingly, when an absorptive wavelength and antioxidant abilities are identified from blue light wavelength, damage to skin cells may be effectively prevented.

The disclosure of this section is to provide background information relating to the invention. Applicant does not admit that any information contained in this section constitutes prior art.

The following patent documents and non-patent documents disclose related technologies.

### Patent Document

Korean Patent Laid-Open Publication No. 10-2019-0121956
Korean Patent Registration No. 10-1834872

### Non-Patent Document

Liebmann J., et al., J. Invest. Dermatol. 130(1), 259-269 (2010)
Nakashima Y., et al., Free Radic. Bio. Med. 108, 300-310 (2017)
Godley B F. et al., J. Biol. Chem. 280(22), 21061-21066 (2005)
Kang, S., et al., Am. J. Pathol., 166, 1691-1699(2005)
Matsumura, Y., at al., Toxicol. Appl. Pharmacol., 195(3), 298-308(2004)
Simon R. et al., 49, 1603-1616(2010)
Afaq F., et al., Experimental Dermatology, 15, 678-684 (2006)
Anandakumar S et al., J. Korean Soc Food SciNutr 43(4), 612-617(2014)

### SUMMARY

The present disclosure relates to a cosmetic composition for blocking blue light, which contains Curcuma aromatica Salisb extract and Elaeocarpus sylvestris var. ellipticus extract as effective ingredients, as well as a pharmaceutical composition for prevention, improvement or treatment of light-derived skin cell damage, which contains Curcuma aromatica Salisb extract and Elaeocarpus sylvestris var. ellipticus extract as effective ingredients. More particularly, the present disclosure relates to a blue light blocking composition and a pharmaceutical composition for prevention, improvement or treatment of light-derived skin cell damage due to blue light, each of which contains Curcuma aromatica Salisb extract and/or Elaeocarpus sylvestris var. ellipticus extract as effective ingredients, so as to inhibit, prevent, improve or treat the light-derived skin cell damage caused by blue light emitting from different light sources.

One aspect of the present invention provides a cosmetic composition for blocking blue light, which includes Curcuma aromatica Salisb extract or both of Curcuma aromatica Salisb extract and Elaeocarpus sylvestris var. ellipticus extract, a pharmaceutical composition for prevention, improvement or treatment of light-derived skin cell damage, which includes Curcuma aromatica Salisb extract or both of Curcuma aromatica Salisb extract and Elaeocarpus sylvestris var. ellipticus extract, as well as a health supplement food composition for prevention or improvement of light-derived skin cell damage, which includes Curcuma aromatica Salisb extract or both of Curcuma aromatica Salisb extract and Elaeocarpus sylvestris var. ellipticus extract.

Another aspect of the present invention provides a cosmetic composition for blocking blue light, which includes Curcuma aromatica Salisb extract or both of Curcuma aromatica Salisb extract and Elaeocarpus sylvestris var. ellipticus extract.

Still another aspect of the present invention provides a pharmaceutical composition for prevention, improvement or treatment of light-derived skin cell damage, which includes Curcuma aromatica Salisb extract or both of Curcuma aromatica Salisb extract and Elaeocarpus sylvestris var. ellipticus extract, or a health supplement food composition for prevention or improvement of light-derived skin cell damage, which includes Curcuma aromatica Salisb extract or both of Curcuma aromatica Salisb extract and Elaeocarpus sylvestris var. ellipticus extract.

A further aspect of the present invention provides a method for preparation of such Curcuma aromatica Salisb extract and/or Elaeocarpus sylvestris var. ellipticus extract.

As used herein, blue light is one type of visible lights, which has a short wavelength of 380 to 500 nm, and refers to a light source of blue color emitted from computer monitors, smartphones, TVs and the like.

Light-derived skin cell damage as used herein refers to a damage to the skin caused by exposure to sunlight, and may include a damage caused by ultraviolet rays, infrared rays, visible lights including blue light, UV, etc., and in particular a blue light-derived damage.

The light-derived skin cell damage may include, for example, skin thinning, skin atrophy, decrease of collagen fibers and/or elastic fibers, elastosis, loss of skin elasticity, dryness, wrinkle formation, inflammatory cells infiltration, premature skin aging, vascular change, pigmentary change, comedone, cyst and skin pathologic change, but it is not limited thereto.

When the pharmaceutical composition for prevention, improvement or treatment of light-derived skin cell damage according to embodiments of the present invention, which contains Curcuma aromatica Salisb extract and/or Elaeocarpus sylvestris var. ellipticus extract, includes the Curcuma aromatica Salisb extract and Elaeocarpus sylvestris var. ellipticus extract in an alone or mixed form, these components may be included in an amount of 0.1 to 99% by weight (“wt. %”), and in one embodiment, 0.1 to 5 wt. % based on a total weight of the pharmaceutical composition. If the amount is less than the above range, effects of preventing, improving or treating the light-derived skin cell damage are insignificant, and if the amount exceeds the above range, toxicity may be caused.

In addition, when including Curcuma aromatica Salisb extract and Elaeocarpus sylvestris var. ellipticus extract in a mixed form, the Curcuma aromatica Salisb extract and the Elaeocarpus sylvestris var. ellipticus extract are included in a weight ratio of 1:1 to 3, and in embodiments, 1:2. If the weight ratio is not within the above range, functional effects may be reduced.

The pharmaceutical composition according to embodiments of the present invention may be administered in oral or non-oral route. In a case of non-oral administration, the composition may be administered in a method such as intravenous, subcutaneous, intramuscular, intraperitoneal, transdermal, intramucosal and ophthalmic administrations, local administration, or the like, but it is not limited thereto.

A proper dose for administration of the pharmaceutical composition according to embodiments of the present invention may depend upon different prescriptions in consideration of formulation methods, administration manners, age, weight, gender or pathogenic conditions of a patient, food intake, administration time, administration route, excretion rate, reaction sensitivity, or the like. The administration dose of the pharmaceutical composition according to embodiments of the present invention may range from 0.0001 to 100 mg/kg (of body weight) on the basis of an adult, but it is not limited thereto.

The pharmaceutical composition described above may further include any suitable carrier, excipient and/or diluent commonly used for production in the art.

Pharmaceutically acceptable carriers, excipients and/or diluents possibly included in the pharmaceutical composition according to an embodiment of the present invention may include, for example, lactose, dextrose, sucrose, calcium silicate, cellulose, methyl cellulose, amorphous cellulose, polyvinyl pyrrolidone, water, methyl hydroxybenzoate, propyl hydroxybenzoate, talc, magnesium stearate and mineral oil, but it is not limited thereto. The pharmaceutical composition according to embodiments of the present invention may be formulated into diverse formulations such as gels, tablets, capsules, granules, suspensions, syrups, etc. by any conventional method known in the art, to which the present invention pertains, for the convenience of use.

When the cosmetic composition for blocking blue light, which contains Curcuma aromatica Salisb extract and/or Elaeocarpus sylvestris var. ellipticus extract, includes the Curcuma aromatica Salisb extract and Elaeocarpus sylvestris var. ellipticus extract in an alone or mixed form, these components may be included in an amount of 0.1 to 99 wt. %, and in one embodiment, 0.1 to 5 wt. % based on a total weight of the cosmetic composition. If the amount is less than the above range, effects of preventing, improving or treating a light-derived skin cell damage are insignificant, and if the amount exceeds the above range, toxicity may be derived.

Further, when including the Curcuma aromatica Salisb extract and the Elaeocarpus sylvestris var. ellipticus extract in a mixed form, a weight ratio of the Curcuma aromatica Salisb extract:the Elaeocarpus sylvestris var. ellipticus extract may be 1:1 to 3, and in one embodiment, 1:2. If the weight ratio is not within the above range, functional effects may be reduced.

In addition, the cosmetic composition may further include antioxidants, stabilizers, solvates, vitamins, etc., which are commonly used for preparation of cosmetics, typical supplemental agents such as pigments and flavoring agents, and carriers. Further, the inventive composition may be prepared into a variety of formulations including, for example, basic cosmetics such as toilet water, essences, creams, packs, foundations, makeup bases, etc., color cosmetics and hair products or the like. Specifically, the inventive composition may be applied in different states such as liquid, cream, paste, solid and the like. In addition, the inventive composition may be prepared by conventional methods for preparing cosmetics.

When the food composition for prevention or improvement of light-derived skin cell damage, which contains Curcuma aromatica Salisb extract and/or Elaeocarpus sylvestris var. ellipticus extract, includes the Curcuma aromatica Salisb extract and Elaeocarpus sylvestris var. ellipticus extract in an alone or mixed form, these components may be included in an amount of 0.1 to 99 wt. %, and in one embodiment, 0.1 to 5 wt. % based on a total weight of the food composition. If the amount is less than the above range, effects of preventing, improving or treating a light-derived skin cell damage are insignificant. If the amount is more than the above range, toxicity may be derived.

Further, when including the Curcuma aromatica Salisb extract and the Elaeocarpus sylvestris var. ellipticus extract in a mixed form, a weight ratio of the Curcuma aromatica Salisb extract:the Elaeocarpus sylvestris var. ellipticus extract may be 1:1 to 3, and in one embodiment, 1:2. If the weight ratio is not within the above range, functional effects may be reduced.

The food composition containing Curcuma aromatica Salisb extract and/or Elaeocarpus sylvestris var. ellipticus extract according to embodiments of the present invention may be prepared into a variety of formulations including, for example, beverage, gum, tea, vitamin complex, powder, granules, tablets, capsules, confectionary and the like. Further, the food composition may also include a health supplement food composition, and additionally include additives commonly used in the art, such as thickeners, stabilizers, flavoring agents, pH adjusters, colorant and the like. In addition, the food composition may be prepared by any conventional method used for food production in the art.

The Curcuma aromatica Salisb extract and/or the Elaeocarpus sylvestris var. ellipticus extract according to embodiments of the present invention may be prepared by a preparing method including the following steps of:

(a) performing extraction using a solvent;

(b) adding at least one selected from the group consisting of carbohydrate hydrolysis enzymes, pectin hydrolysis enzymes and cellulose hydrolysis enzymes to the extract obtained in step (a), followed by stirring and incubating the same; and

(c) adding an organic solvent for fractionalization to the culture product in step (b), followed by fractionalizing the same.

Herein, the solvent in step (a) may include at least one selected from the group consisting of alcohol having 1 to 4 carbon atoms or an aqueous alcohol solution, such as ethanol solution, methanol solution, isopropanol solution, butanol solution, etc. In embodiments, the ethanol solution is used in consideration of a treatment process using the enzyme in the subsequent step of step (b) or toxicity, but not limited thereto.

Further, in one embodiment, the aqueous alcohol solution has an alcohol content of about 50 wt. % to 100 wt. %, and in another embodiment, about 75 wt. % to 95 wt. %.

In one embodiment, the extraction in step (a) is conducted at 60 to 90° C. If the temperature is too low, an extraction yield is deteriorated and an extraction time is extended. If extraction is conducted at an extremely high temperature, the effective ingredients may be denatured.

Step (b) described above may include adding at least one selected from the group consisting of carbohydrate hydrolysis enzymes, pectin hydrolysis enzymes and cellulose hydrolysis enzymes to the extract obtained in step (a), followed by stirring and incubating the same. In embodiments, an incubation temperature ranges from 40 to 70° C., but not limited thereto.

In order to increase efficiency of step (b) using enzymes, in one embodiment, the preparing method may include step (a′) of concentrating the extract in step (a) and then dissolving the concentrated product in an ethanol solution between steps (a) and (b), wherein the ethanol solution may have an ethanol content of 5 to 30 wt. %, which is lower than that in the extraction of step (a), and in one embodiment, about 10 wt. %.

Step (c) may include adding an organic solvent for fractionalization to the culture product in step (b) to fractionalize the same, wherein the organic solvent for fractionalization may include ethyl acetate, diethyl ether, trichloromethane, butanol, which are used alone or in a mixed form.

The cosmetic composition for blocking blue light, which includes Curcuma aromatica Salisb extract and/or Elaeocarpus sylvestris var. ellipticus extract, may absorb blue light to block the same, so as to inhibit induction of damage to human fibroblasts and eliminate reactive oxygen species. Accordingly, since cell apoptosis is reduced while recovering cell viability to exhibit blue light blocking effects, Curcuma aromatica Salisb extract or a composite of Curcuma aromatica Salisb extract and Elaeocarpus sylvestris var. ellipticus extract according to embodiments of the present invention may be effectively used as a raw material for a pharmaceutical composition for prevention, improvement or treatment of light-derived skin cell damage due to blue light, a cosmetic composition for blocking blue light, and a health supplement food composition for prevention or improvement of light-derived damage.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a graph illustrating absorbance of Curcuma aromatica Salisb extract according to one embodiment of the present invention at a wavelength of 380 to 500 nm, which is a blue light wavelength range;

FIG. 2 is a graph illustrating transmittance of Curcuma aromatica Salisb extract according to one embodiment of the present invention at a wavelength of 380 to 500 nm, which is a blue light wavelength range;

FIG. 3 is a graph illustrating cell viability depending on concentrations of Curcuma aromatica Salisb extract and a mixture of Curcuma aromatica Salisb extract and Elaeocarpus sylvestris var. ellipticus extract according one embodiment of the present invention;

FIG. 4 is a graph illustrating cell viability when treating fibroblasts with Curcuma aromatica Salisb extract according to one embodiment of the present invention, followed irradiating the same with blue light;

FIG. 5 is a graph illustrating cell viability when treating fibroblasts with 25 μg/ml of Curcuma aromatica Salisb extract and Elaeocarpus sylvestris var. ellipticus extract, simultaneously, according to one embodiment of the present invention, followed irradiating the same with blue light;

FIG. 6 is a graph illustrating effects of inhibiting ROS generation in cells induced by irradiation of blue light by Curcuma aromatica Salisb extract according to one embodiment of the present invention; and

FIG. 7 is a graph illustrating results of lipid peroxidation of Curcuma aromatica Salisb extract, and a mixture of Curcuma aromatica Salisb extract and Elaeocarpus sylvestris var. ellipticus extract according to one embodiment of the present invention.

### DETAILED DESCRIPTION OF EMBODIMENTS

Hereinafter, the present invention will be described in detail by way of embodiments, examples and experimental examples. However, such examples or experimental examples according to the present invention may be modified into various and different forms, and a scope of the present invention should not be construed as being limited to the following examples. The examples and experimental examples according to the present invention are only provided for more completely describing the present invention to a person having ordinary knowledge in the art to which the present invention pertains. Therefore, it should be understood that various modifications and equivalents replacing the above embodiments at a time of filing the present application are possibly included.

Curcuma aromatica Salisb is a perennial plant belonging to Zingiberaceae, which is originated from India and is known as a natural spice affording yellow color to food. Major components of this plant include about 70% of carbohydrate and curcuminoids, and, among them, about 90% of curcumin, which is a polyphenol component, is contained therein.

Elaeocarpus sylvestris var. ellipticus is a plant growing wild in Jeju Island and contains various ingredients such as pentagalloyl glucose (PGG), coniferylalcohol, umbelliferone, scopoletin, (3-sitosterol, daucosterol, etc. Further, Elaeocarpus sylvestris var. ellipticus tree is known to have a variety of physiological activities such as anti-diabetic, anti-oxidative, anti-inflammatory, anti-cholesterol, anti-viral and anti-bacterial activities, etc.

In one example, a pharmaceutical composition for inhibiting virus includes Elaeocarpus sylvestris var. ellipticus extract as effective ingredients.

However, a cosmetic composition for blocking blue light, which includes or uses Curcuma aromatica Salisb extract or a composite of Curcuma aromatica Salisb extract and Elaeocarpus sylvestris var. ellipticus extract, is not yet known. Accordingly, the present inventors found that Curcuma aromatica Salisb extract or a composite of Curcuma aromatica Salisb extract and Elaeocarpus sylvestris var. ellipticus extract absorbs blue light having a wavelength of 380 to 500 nm, and does not transmit the same but exhibits effects of inhibiting free radical formation.

Examples of the present invention are discussed below.

### Example 1. Preparation of Extract

After crushing 50 g of dried Curcuma aromatica Salisb and 50 g of Elaeocarpus sylvestris var. ellipticus, 500 ml of 95% ethanol solution was added, followed by heating for extraction at 60 to 90° C. and filtration. This extracting process was repeated twice, followed by concentration to obtain each extract. The extracts were dissolved in 100 mg/ml of dimethyl sulfoxide (DMSO) to prepare a stock solution to be used.

### Experimental Example 1. Evaluation of Blue Light Absorption and Transmission of Curcuma aromatica Salisb Extract and Elaeocarpus sylvestris Var. Ellipticus Extract

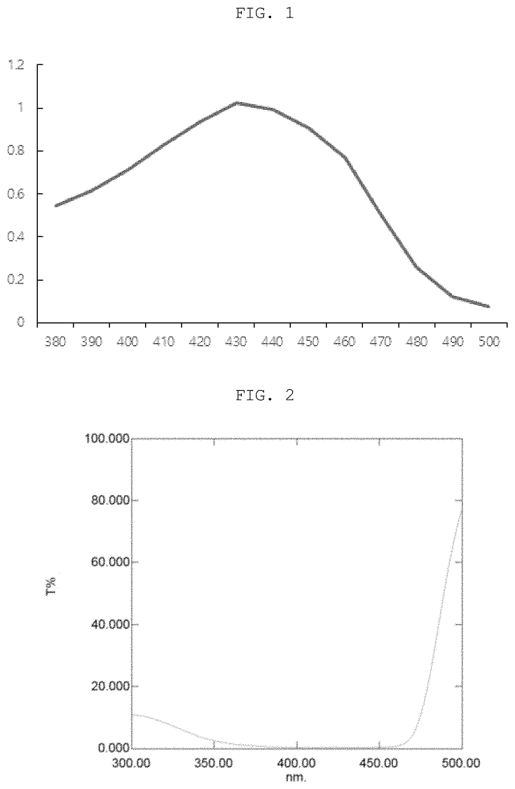

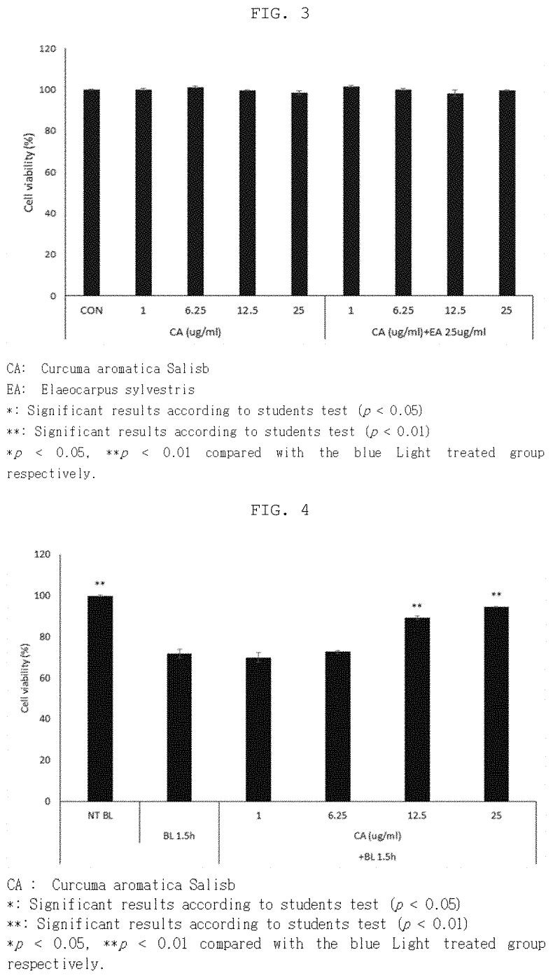

In order to determine changes in blue light absorption and transmission of Curcuma aromatica Salisb extract, absorbance and transmittance were measured at a wavelength of 380 to 500 nm which is a blue light wavelength range, and results thereof are shown in FIGS. 1 and 2.

As a result of the measurement, it can be seen that the Curcuma aromatica Salisb extract according to one embodiment of the present invention might absorbs a blue light at the wavelength of 380 to 500 nm which is the blue light wavelength range. Specifically, maximum absorbance was shown at 430 nm (FIG. 1).

Further, it can be seen that the Curcuma aromatica Salisb extract according to one embodiment of the present invention almost absorbs blue light having a wavelength of 380 to 460 nm to prevent transmission of blue light (FIG. 2).

### Experimental Example 2. Assessment of Antioxidant Effects of Curcuma aromatica Salisb Extract and Elaeocarpus sylvestris Var. Ellipticus Extract

In order to assess antioxidant effects of the Curcuma aromatica Salisb extract and the Elaeocarpus sylvestris var. ellipticus extract according to one embodiment of the present invention, free radical scavenging activity test was implemented. Such free radical scavenging activity test is a modification of Kim et al. method (Kor. J. Pharmacogn., 24 (4), 99-303(1993)), wherein, as a stable free radical, 1,1-diphenyl-2-picrylhydrazyl radical (DPPH) (Sigma) was used.

Firstly, the extraction sample obtained in the above example was diluted in methanol at a proper concentration was mixed in 1 ml of 0.2 mM DPPH solution to prepare 2 ml of diluted sample, and left at room temperature for 10 minutes, followed by measuring absorbance at 517 nm.

Meanwhile, instead of the sample solution, a control in the free radical scavenging test was prepared by adding methanol, followed by performing a measurement by the same method as the above experimental example. Instead of DPPH solution, methanol was added to afford each color compensated value for each of the extraction sample and the control.

Using the following Equation 1, free radical scavenge rates were calculated and listed in Table 2 below as numeral values. In Table 2 below, SC50 refers to a concentration of sample used for achieving 50% of free radical scavenge rate, which is a numeral value used for comparison relative to other constitutional substances, and indicates that a smaller the value, a higher the scavenge rate.

$$Free radical scavange rate ( \% ) = 100 - \frac{Absorbance of each extraction sample}{Absorbance of control} \times 100 \qquad [ Equation 1 ]$$

TABLE 2

| | Free radical scavenge ability |
|---|---|
| Extraction sample | SC₅₀ (ug/ml) |
| Curcuma aromatica Salisb | 43.6 ± 1.3 |
| extract | |
| Elaeocarpus sylvestris | 16.4 ± 0.2 |
| var. Ellipticus extract | |

As a result of the experiment, it confirmed that each of the Curcuma aromatica Salisb extract and the Elaeocarpus sylvestris var. ellipticus extract prepared in the examples had excellent antioxidant activity.

### Experimental Example 3. Measurement of Cytotoxicity

Lactate dehydrogenase assay is a method for measuring an amount of LDH in cytoplasm leaking out of cells. Existence of LDH in a cell culture medium represents a damage of a cellular wall, and through this, it is possible to identify an extent of cytotoxicity. Lactate dehydrogenase activity measurement was performed by using lactate dehydrogenase (LDH) assay provided by Dojindo Lab (Tokyo Japan). Briefly, after setting an amount of HS68 cell lines to 1×10⁵ cells/ml, each 100 μl was dispensed into a 96-well plate, followed by incubation in CO₂ incubator for 24 hours. Thereafter, the cell line was treated with each of samples at different concentrations and then cultured. Each 50 μl supernatant of the culture solution was dispensed into a new 96-well plate, followed by adding each 50 μl of LDH reagent thereto and conducting a reaction. Thereafter, when the reaction is completed, each 100 μl of stop solution was added thereto to terminate the reaction. The product was subjected to measurement at 490 nm using a microplate reader, and results thereof are shown in FIG. 3.

As a result of the experiment, it could be seen that, when the product was treated with each of the Curcuma aromatica Salisb extract and the Elaeocarpus sylvestris var. ellipticus extract at each concentration of 1, 6.25, 12.5, 25 μg/ml in order to determine cytotoxicity to the above extracts, LDH values were substantially not changed as compared to a control which was treated with DMSO only.

### Experimental Example 4. Assessment of Cell Protective Effects of Curcuma aromatica Salisb Extract Against Blue Light

Cell protective effects of the Curcuma aromatica Salisb extract against blue light were assessed by using the lactate dehydrogenase activity measurement method. Human fibroblast HS68 was incubated in a 96 well plate. A sample was treated and irradiated with a blue light having a certain wavelength for 1.5 hours. Thereafter, each 50 μl supernatant of the culture solution was dispensed into a new 96-well plate, followed by adding each 50 μl of LDH reagent and conducting a reaction. Thereafter, when the reaction is completed, each 100 μl of stop solution was added thereto to terminate the reaction. The product was subjected to measurement at 490 nm using a microplate reader. Cell protective effects were defined as a percentage relative to an average absorbance of each control.

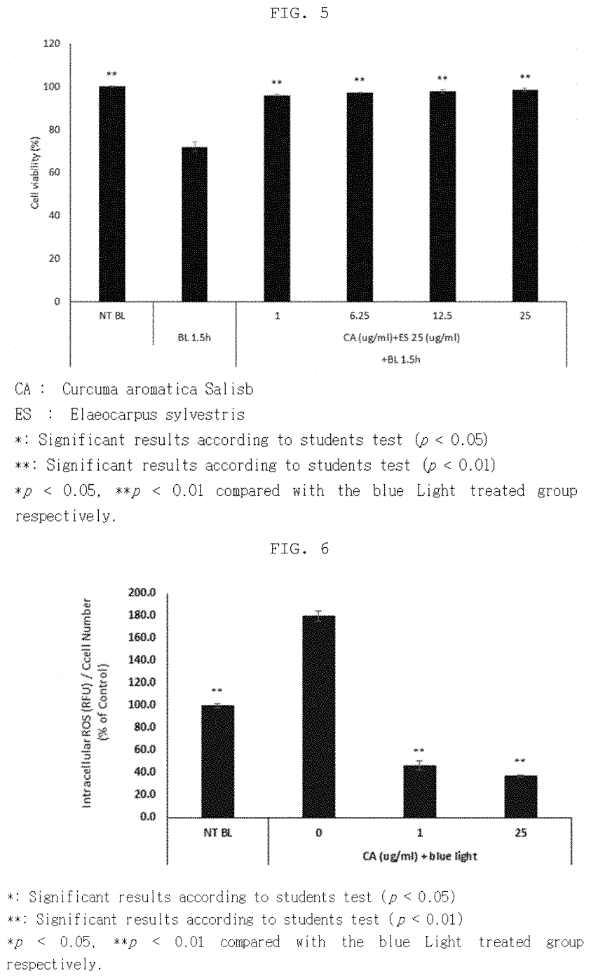

In order to assess cell protective effects of the Curcuma aromatica Salisb extract against blue light in fibroblasts, when the cells were treated with the extract at each concentration of 1, 6.25, 12.5 and 25 μg/ml, an extent of change in cell viability was calculated as compared to a control, which was treated with DMSO only, so as to assess cell protection. Results thereof are shown in FIG. 4. As a result of the experiment, it confirmed that the Curcuma aromatica Salisb extract exhibited cell protective effects against blue light at each concentration of 1, 6.25, 12.5 and 25 μg/ml (FIG. 4).

Further, when treating the cells with both of the Curcuma aromatica Salisb extract and the Elaeocarpus sylvestris var. ellipticus extract, simultaneously, as well as when treating the cells with the Elaeocarpus sylvestris var. ellipticus extract alon, it confirmed that cell protective effects against blue light was increased (FIG. 5).

### Experimental Example 5. Determination of Reactive Oxygen Species (ROS) in Cells

In order to determine ROS in skin cells irradiated with blue light, the skin cells were treated with Curcuma aromatica Salisb extract and irradiated with a blue light, followed by adding DMEM medium added with 100 μM DCF-DA. After 30 minutes, DCF was measured. The cultured cells were incubated in a DMEM medium added with DCFH-DA in 5% CO₂ incubator at 37° C. for 30 minutes, and DCFH-DA was washed with HBSS three times. Then, fluorescence was measured at an excitation wavelength of 485 nm and an emission wavelength of 530 nm by using a fluorescence microplate reader (Bio-Tek instrument, USA)

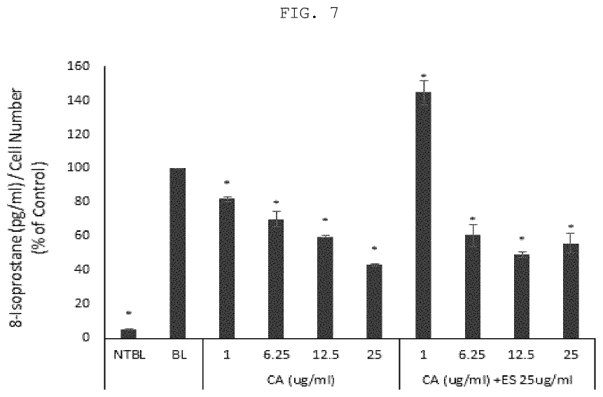

As shown in FIG. 6, ROS in cells was significantly inhibited due to the treatment of Curcuma aromatica Salisb extract at each concentration of 1 and 25 μg/ml. Therefore, it is proposed that the Curcuma aromatica Salisb extract removes ROS generated due to blue light to exhibit skin protective effects against blue light.

### Experimental Example 6. Lipid Peroxidation Assay

In order to determine lipid peroxidation, an amount of 8-isoprostane secreted from fibroblasts in a cell culture solution was determined by using an enzyme immunoassay kit commercially available in the market (enzyme immunoassay, Cayman Chemical, Ann Arbor, Mich., USA) through colorimetric determination according to an instruction of the manufacturer.

In order to identify whether Curcuma aromatica Salisb extract and Elaeocarpus sylvestris var. ellipticus extract may inhibit peroxidation of membrane lipid in cells irradiated with a blue light, the sample was treated and measured after irradiation with the blue light. Lipid peroxidation was identified by measuring an amount of 8-isoprostane in skin cell culture solution. Consequently, as shown in FIG. 7, it confirmed that a level of increased 8-isoprastane in cells exposed to blue light was significantly reduced depending on the concentration due to the treatment with Curcuma aromatica Salisb extract or a mixture of Curcuma aromatica Salisb extract and Elaeocarpus sylvestris var. ellipticus extract. Consequently, as shown in FIGS. 4, 5 and 6, reactive oxygen in the cells was reduced by the Curcuma aromatica Salisb extract and the Elaeocarpus sylvestris var. ellipticus extract, and therefore, as shown in FIG. 7, lipid peroxidation was inhibited. Accordingly, it can be seen that Curcuma aromatica Salisb extract and Elaeocarpus sylvestris var. ellipticus extract exhibit effects of treating, improving or preventing a light-derived skin cell damage due to blue light.

As described above, embodiments of the present invention provide a natural material-based composition with excellent blue light blocking effects, which contains Curcuma aromatica Salisb extract and Elaeocarpus sylvestris var. ellipticus extract as effective ingredients, and therefore, is very available in bio-technical, foods, cosmetics and medical industries.

What is claimed is:

1. A cosmetic composition for blocking blue light, comprising Curcuma aromatica Salisb extract and/or Elaeocarpus sylvestris var. ellipticus extract.

2. The cosmetic composition according to claim 1, wherein the Curcuma aromatica Salisb extract and/or the Elaeocarpus sylvestris var. ellipticus extract are/is included in an amount of 0.1 to 99% by weight (“wt. %”) based on a total weight of the cosmetic composition.

3. The cosmetic composition according to claim 1, wherein a weight ratio of the Curcuma aromatica Salisb extract:the Elaeocarpus sylvestris var. ellipticus extract is 1:1 to 3.

4. A pharmaceutical composition for prevention, improvement or treatment of light-derived skin cell damage, comprising Curcuma aromatica Salisb extract and/or Elaeocarpus sylvestris var. ellipticus extract.

5. The pharmaceutical composition according to claim 4, wherein the Curcuma aromatica Salisb extract and/or the Elaeocarpus sylvestris var. ellipticus extract are/is included in an amount of 0.1 to 99 wt. % based on a total weight of the pharmaceutical composition.

6. The pharmaceutical composition according to claim 4, wherein a weight ratio of the Curcuma aromatica Salisb extract:the Elaeocarpus sylvestris var. ellipticus extract is 1:1 to 3.

7. The pharmaceutical composition according to claim 4, wherein the light-derived skin cell damage is a skin cell damage due to blue light having a wavelength of 380 to 500 nm.

8. The pharmaceutical composition according to claim 4, wherein the light-derived skin cell damage is any one or more among skin thinning, skin atrophy, decrease of collagen fibers and/or elastic fibers, elastosis, loss of skin elasticity, dryness, wrinkle formation, inflammatory cells infiltration, premature skin aging, vascular change, pigmentary change, comedone, cyst and skin pathologic change.

9. A health supplement food composition for prevention or improvement of light-derived skin cell damage, comprising Curcuma aromatica Salisb extract and/or Elaeocarpus sylvestris var. ellipticus extract.

10. The health supplement food composition according to claim 9, wherein the Curcuma aromatica Salisb extract and/or the Elaeocarpus sylvestris var. ellipticus extract are/is included in an amount of 0.1 to 99 wt. % based on a total weight of the food composition.

11. The health supplement food composition according to claim 9, wherein a weight ratio of the Curcuma aromatica Salisb extract:the Elaeocarpus sylvestris var. ellipticus extract is 1:1 to 3.

12. The health supplement food composition according to claim 9, wherein the light-derived skin cell damage is a skin cell damage due to blue light having a wavelength of 380 to 500 nm.

13. The health supplement food composition according to claim 9, wherein the light-derived skin cell damage is any one or more among skin thinning, skin atrophy, decrease of collagen fibers and/or elastic fibers, elastosis, loss of skin elasticity, dryness, wrinkle formation, inflammatory cells infiltration, premature skin aging, vascular change, pigmentary change, comedone, cyst and skin pathologic change.

14. A method for preparing the cosmetic composition comprising Curcuma aromatica Salisb extract and/or the Elaeocarpus sylvestris var. ellipticus extract according to claim 1, comprising:
(a) extracting Curcuma aromatica Salisb and/or Elaeocarpus sylvestris var. ellipticus with alcohol having 1 to 3 carbon atoms or an aqueous alcohol solution;
(b) adding at least one selected from the group consisting of carbohydrate hydrolysis enzymes, pectin hydrolysis enzymes and cellulose hydrolysis enzymes to the extract obtained in step (a), followed by stirring and incubating the same; and
(c) adding an organic solvent for fractionalization to the culture product in step (b) to fractionalize the same.

15. A method for preparing the pharmaceutical composition comprising Curcuma aromatica Salisb extract and/or the Elaeocarpus sylvestris var. ellipticus extract according to claim 4, comprising:
(a) extracting Curcuma aromatica Salisb and/or Elaeocarpus sylvestris var. ellipticus with alcohol having 1 to 3 carbon atoms or an aqueous alcohol solution;
(b) adding at least one selected from the group consisting of carbohydrate hydrolysis enzymes, pectin hydrolysis enzymes and cellulose hydrolysis enzymes to the extract obtained in step (a), followed by stirring and incubating the same; and
(c) adding an organic solvent for fractionalization to the culture product in step (b) to fractionalize the same.

16. A method for preparing the health supplement food composition comprising Curcuma aromatica Salisb extract and/or the Elaeocarpus sylvestris var. ellipticus extract according to claim 9, comprising:
(a) extracting Curcuma aromatica Salisb and/or Elaeocarpus sylvestris var. ellipticus with alcohol having 1 to 3 carbon atoms or an aqueous alcohol solution;
(b) adding at least one selected from the group consisting of carbohydrate hydrolysis enzymes, pectin hydrolysis enzymes and cellulose hydrolysis enzymes to the extract obtained in step (a), followed by stirring and incubating the same; and
(c) adding an organic solvent for fractionalization to the culture product in step (b) to fractionalize the same.

17. A method of preventing, improving or treating light-derived skin cell damage using the composition of claim 4, the method comprising:
administering the composition of claim 4 to a patient in an oral or non-oral route.

18. A method of preventing, improving or treating light-derived skin cell damage using the composition of claim 4, the method comprising:
applying the composition of claim 4 on a skin of a patient.
